(19) United States
(12) Patent Application Publication
(10) Pub. No.: US 2006/0128675 A1
(43) Pub. Date: Jun. 15, 2006

(54) Method for monitoring the effectiveness of teracycline in the treatment of asthma

(76) Inventors: Helen G Durkin, Manhasset, NY (US); Rauno Joks, Port Washington, NY (US); Tamar A. Smith-Norowitz, Brooklyn, NY (US); Martin H Bluth, West Hempstead, NY (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/523,856

(22) PCT Filed: Aug. 8, 2003
(86) PCT No.: PCT/US03/24746

§ 371 Date: Oct. 18, 2005

(30) Foreign Application Priority Data
Aug. 9, 2002 (US) 60402411

(51) Int. Cl. A61K 31/65

(52) U.S. Cl. 514/152; 514/154

(57) ABSTRACT

The present invention is directed to a method of lowering excess IgE levels in a mammal suffering from a disease where IgE is pathogenic which method comprises administering to said mammal an IgE lowering effective amount of a tetracycline such as minocycline or doxycline. It is also directed to a method of monitoring the effectiveness of a drug in lowering the concentration of excess IgE in plasma in a mammal suffering from the disease in which IgE is pathogenic which method comprises making a first determination of the concentration of IgE in the plasma of said mammal at an initial time; administering to said mammal the drug; making a second determination of the concentration of IgE in the plasma of said mammal after the initial time and after administration of the drug and comparing the values obtained from the first and second determinations, such that if the value of the second determination is higher than or about the same as the value of the first determination and above a threshold level, then the dosage amount of the drug administered to the mammal is increased; otherwise the dosage amount is maintained.

### BACKGROUND OF THE INVENTION

This invention relates to the discovery that minocycline and doxycline, and other tetracyclines are effective in lowering IgE levels in mammals, especially humans, suffering from a disease where IgE is pathogenic, such as allergies, asthma, especially human allergic response, and diseases associated with an inflammatory response.

Diseases involving inflammation are characterized by the influx of certain cell types and mediators, the presence of which can lead to tissue damage and sometimes death. Diseases involving inflammation are particularly harmful when they afflict the respiratory system, resulting in obstructed breathing, hypoxemia, hypercapnia and lung tissue damage. Obstructive diseases of the airways are characterized by air flow limitation (i.e., airflow obstruction or narrowing) due to constriction of airway smooth muscle, edema and hypersecretion of mucous leading to increased work in breathing, dyspnea, hypoxemia and hypercapnia.

A variety of inflamatory agents can provide air flow limitation, such as for example, allergens. In particular, allergens and other agents in allergic or sensitized animals (i.e., antigens and haptens) cause the release of inflammatory mediators that recruit cells involved in inflammation. Such cells include lymphocytes, eosinophils, mast cells, basophils, neutrophils, macrophages, monocytes, fibroblasts and platelets. A variety of studies have linked the degree, severity and timing of the inflammatory process with the degree of airway hyperresponsiveness. Thus, a common consequence of inflammation is airflow limitation and/or airway hyperresponsiveness. Asthma is a significant disease of the lung which is typically characterized by periodic air flow limitation and/or hyperresponsiveness to various stimuli which results in excessive airways narrowing. Other characteristics can include inflammation of airways and eosinophila. More particularly, allergic asthma is often characterized by eosinophilic airway inflammation arid airway responsiveness.

An estimated 16 million persons in the U.S. have asthma, which is about 10% of the population. The numbers have increased about 25% in the last 20 years. The estimated cost of treating asthma in the U.S. exceeds $6 billion. About 25% of patients with asthma who seek emergency care require hospitalization. The largest single direct medical expenditure for asthma has been in patient hospital services (emergency care), at a cost of greater than $1.6 billion. The cost for prescription medications is at least $1.1 billion.

According to the National Ambulatory Medical Care Survey, asthma accounts for 1% of all ambulatory care visits and the disease continues to be a significant cause of missed school days in children. Despite improved understanding of the disease process and better drugs, asthma morbidity and mortality continues to rise in this country and worldwide. Thus, asthma constitutes a significant public health problem.

The pathophysiologic processes that attend the onset of an asthmatic episode can be broken down into essentially two phases, both marked by bronchioconstriction, that causes wheezing, chest tightness, and dyspnea. The first, early phase asthmatic response is triggered by allergens and irritants. Allergens cross-link immunoglobulin (IgE) molecules bound to receptors on mast cells, causing them to release a number of pre-formed inflammatory mediators, including histamine. Additional triggers include the osmotic changes in airway tissues following exercise and/or the inhalation of cold, dry air. The second, late phase response that follows is characterized by infiltration of activated eosinophilis and other inflammatory cells into airway tissues, epithelial desquamonon and by the presence of highly viscous mucus within the airway. The damage caused by this inflammatory response leaves the airways “primed” or sensitized, such that smaller triggers are required to elicit subsequent asthma symptoms.

For instance, human allergic asthma, a disease characterized by airway hyperresponsiveness and bronchial inflammation, is mediated by a variety of activated leukocytes, including eosinophils, mast cells, CD4+T lymphocytes, and CD 19+β cells.

Current treatments, which improve airway hyperresponsivenss, include various anti-inflammatory agents, which reduce mucosal inflammation and asthma pathogenesis; however their efficacies vary markedly.

Short acting β₂-adrenegric agonists, terbutaline and albuterol, long the mainstay of asthma treatment, act primarily during the early phase as bronchodilators. The newer long acting β₂ agonists do not possess significant anti-inflammatory activity; they have no effect on bronchial hyperreactivity.

Numerous other drugs target specific aspects of the early or late asthmatic responses. For example, antihistamines, like loratadine, inhibit early histamine-mediated inflammatory responses. Other antihistamines, such as azelastine and ketotifen, have both anti-inflammatory and weak bronchodilatory effects, but they currently do not have any established efficacy in asthma treatment.

Phosphodiesterase inhibitors, like theophylline/xanthines, may attenuate late inflammatory responses, but there is no evidence that the compounds decrease bronchial hyperreactivity. Anticholinergics, like ipratopium bromide, which are used in cases of acute asthma to inhibit severe bronchoconstruction, have no effect on early or late phase inflammation, no effect on bronchial hyperreactivity and therefore essentially have no role in chronic therapy.

The corticosteroid drugs, like budesonide, are among the most potent anti-inflammatory agents. Inflammatory mediators or release inhibitors, like cromolyn and nedocromil, act by stabilizing mast cells and inhibiting the late phase inflammatory response to allergen. Thus, cromolyn and nedocromil, as well as the corticosteroids, all reduce bronchial hyperactivity by minimizing the sensitivity effect of inflammatory damage to the airways. These anti-inflammatory agents, however, do not produce bronchodilation.

Thus, while numerous drugs are currently available for the treatment of asthma, these compounds are primarily palliative and/or have significant side effects. Moreover, allergen immunotherapy for asthma is limited in efficacy because patients frequently have sensitivities to multiple allergens and can experience immediate hypersensitivity reactions, with resultant decreased adherence to immunotherapy protocols.

Unfortunately, none of the aforementioned drugs target the underlying cause of asthma. Consequently, new therapeutic approaches which target the underlying cause rather than the cascade of symptoms would be highly desirable. The present inventors have searched for the underlying cause of asthmas, especially human allergic asthma. In asthma, CD4+ T cells secrete IL-4, a (Th-)2 type cytokine, which is required for IgE production and which is implicated in airway hyperresponsiveness, as well another cyctokines which increase IgE production. Although a correlation between IgE and airway hyperresponsiveness has been alleged in allergic asthma, a cause and effect relationship has not as yet been established. The present inventors have found that causal relationship and have found that the tetracyclines, such as minocycline and doxycylcine, and the like, suppress the excess concentration of IgE in the blood plasma of patients suffering from human allergic asthma

An aspect of the present invention is directed to the use of minocycline and doxycycline for treatment of asthma.

Both minocycline and doxycycline are known compounds. For example, it has been reported that allergic steroid dependent asthmatic patients treated with an oral administration of minocycline improved their symptoms (A.M. and P.M.), and decreased oral corticosteroid requirements See Joks et al., J. Allergy Clin. Immunol. 1998, 101:562. Additional studies of O'Dell,. et al. in Arthritis Rheum: 1997, 40: 842-848 and Arthritis Rheum:, 1999, 42:1691-1695 have shown that treatment of mild and moderate rheumatoid arthritis (RA) patients with minocycline had no side effects, and appears to be an effective therapy for early RA. Moreover, Yu, et al. in Arthritis Rheum:, 1992, 35: 1150-1155 reported that treatment of dogs with minocycline or doxycycline reduced the severity of osteoarthritis (OA), while studies by Thong, et al. in Clin Exp Immunol., 1979, 35:443-446, have shown that doxycycline and tetracycline inhibit the ability of mice to mount delayed-type hypersensitivity responses. Further, studies in vitro have demonstrated that treatment of human whole blood cultures with minocycline or tetracycline at physiological doses inhibits mitotic responses to phytohemagglutinin (See Ingham, et al., Antimicrob Chemother, 1991, 27: 607-617) and inhibits inducible nitric oxide synthase (iNOS) expression by murine macrophages See Amin, et al., PNAS, 1996, 93: 14014-14019.

The present inventors have found from their investigation a causal relationship between excessive IgE levels and the airway hyperresponsiveness found in asthma The present inventors-have found that drugs which lower excessive circulatory IgE levels in plasma are effective in treating human allergic asthma. In particular the present inventors have found that tetracyclines, such as minocycline and doxycycline suppress excess IgE levels in patients suffering from asthma, allergies, inflammatory conditions, or other diseases where IgE is pathogenic.

### SUMMARY OF THE INVENTION

Thus, the present invention is directed to a method of lowering excess IgE concentration in the plasma of a mammal suffering from a disease where IgE is pathogenic which method comprises administering to a mammal suffering therefrom an IgE lowering effective amount of a tetracycline. In another embodiment, the present invention is directed to a method of lowering IgE concentration in the plasma of a mammal suffering from a disease in which IgE is pathogenic which comprises administering to said mammal suffering therefrom an IgE lowering effective amount of minocycline or doxycycline or a combination thereof Diseases in which the IgE is pathogenic include allergies and asthma, including human allergic asthma and inflammatory conditions. The present inventors have also found that patients, e.g., mammals, such as humans with higher concentrations of IgE in their plasma are more prone to be suffering form diseases where IgE is pathogenic, e.g., allergies and asthma, including human allergic asthma than those having a lower concentration thereof. Thus, by monitoring the IgE concentration in the plasma, the effectiveness of the drug administered to patients who suffer from diseases in which IgE is pathogenic, e.g., allergies, asthma, and the like, which lower IgE concentration can be determined. Thus, another aspect of the present invention is directed to a method of monitoring the effectiveness of a drug in lowering the concentration of IgE in the plasma in a mammal suffering from a disease in which IgE is pathogenic which method comprises making a first determination of the concentration of IgE in the plasma at an initial time in said mammal; administering an effective amount of drug which lowers IgE concentration in the plasma, e.g., a tetracycline, such as minocycline or doxycycline, or the like or combination thereof to said mammal; at a second time subsequent to the initial time, and after the administration of said drug, e.g., a tetracycline, making a determination of the concentration of IgE in the plasma; comparing the values obtained from the first and second determinations, such that if the value of the second determination of the free IgE level in the plasma is higher than or about the same as the first determination thereof and above a threshold level, then the dosage amount of the drug, e.g., the tetracycline, administered to the mammal is increased; otherwise the dosage regimen is maintained.

### BRIEF DESCRIPTION OF THE DRAWING

FIG. 1 illustrates the IgE production in vitro by peripheral blood mononuclear cells (PBMC) from asthmatic and non asthmatic subjects after tetracycline treatment.

### DETAILED DESCRIPTION OF THE INVENTION

As used herein the term “patient” or “subject” refers to a warm blooded animal and preferably mammals, such as for example, cats, dogs, horse, cows, pigs, mice, rats and primates including humans. The preferred patient is humans.

The term “drug” herein is used to connote a compound which is used to lower the IgE concentration in the plasma of a mammal, e.g., human. The preferred drugs are the tetracyclines.

The term “a tetracycline” or “tetracyclines” refers to the broad-spectrum antibiotics which are members of the tetracycline family. Examples include tetracycline, rolitetracycline, oxytetracycline, chlortetracycline, demeclocycline, meclocycline, methacycline, doxycycline and minocycline, and the like. The preferred tetracyclines are doxycycline and minocycline.

This terminology is to be distinguished from the antibiotic “tetracycline”. It is to be understood that when the term “tetracycline” is used herein by itself, that is, without an article (a, an, the) or if used in the singular, it refers to the antibiotic tetracycline.

An embodiment of the present invention is directed to the use of IgE lowering effective amounts of a tetracycline for the suppression of elevated concentrations of IgE in the blood plasma of a patient suffering from a disease in which IgE is pathogenic. In another embodiment, the present invention is directed to the use of IgE lowering effective amounts of minocycline for the suppression of elevated concentrations of IgE in the blood plasma of the patient, e.g., a human suffering from a disease in which IgE is pathogenic, especially human allergic asthma. Another embodiment of the present invention is directed to the use of IgE lowering effective amounts of doxycycline for the suppression of elevated concentrations of IgE in the blood plasma of the patient, e.g., humans, suffering from a disease where IgE is pathogenic especially human allergic asthma In a further embodiment, the present invention is directed to a use of a combination of both minocycline and doxycycline in IgE lowering effective amounts for the suppression of elevated concentrations of IgE in the blood plasma of a patients, e.g., human suffering from disease where IgE is pathogenic, especially human allergic asthma. Accordingly, minocycline or doxycycline or both in combination in amounts effective to lower the concentration of excess IgE concentrations in the plasma are useful for treating human allergic asthma. In addition, the tetracyclines, e.g. minocycline and doxycycline are anti-inflammatory agents and can be used to treat inflammatory conditions when administered to patients in IgE lowering effective amounts, as defined herein.

The tetracyclines are administered therefore in therapeutically effective amounts.

The physician will determine the dosage of the tetracyclines which will be most suitable and it will vary with the form of administration and the particular compound chosen, and furthermore, it will vary depending upon various factors, including but not limited to the patient under treatment, the age of the patient, the severity of the condition being treated and the like. He will generally wish to initiate treatment with small dosages substantially less than the optimum dose of the compound and increase the dosage by small increments until the optimum effect under the circumstances is reached. The tetracyclines, when given orally, are administered in dosages ranging from about 1 to about 400 mg/day and more preferably from about 1 to about 300 mg/day. When given parenterally, the tetracyclines are administered preferably in dosages of, for example, about 1.5 to about 400 mg/day, and more preferably from about 1 to about 300 mg/day also depending upon the host and the severity of the condition being treated and the compound utilized.

This dosage regimen may be adjusted by the physician to provide the optimum therapeutic response. For example, several divided doses may be administered daily or the dose may be proportionally reduced as indicated by the exigencies of the therapeutic situation.

The tetracyclines may be administered in a convenient manner, such as by oral, intravenous (where water soluble), intramuscular or subcutaneous routes.

The tetracyclines may be orally administered, for example, with an inert diluent or with an assimilable edible carrier, or it may be enclosed in hard or soft shell gelatin capsules, or it may be compressed into tablets, or it may be incorporated directly into the food of the diet. For oral therapeutic administration, the tetracyclines may be incorporated with excipients and used in the form of ingestible tablets, buccal tablets, troches, capsules, elixirs, suspensions, syrups, wafers, and the like. Such compositions and preparations should contain at least 1% of the appropriate tetracyclines. The percentage of the compositions and preparations may, of course, be varied and may conveniently be between about 5 to about 80% of the weight of the unit. The amount of the tetracyclines used in such therapeutic compositions is such that a suitable dosage will be obtained. Preferred compositions or preparations according to the present invention contain between about 25 mg and about 1000 mg of a tetracycline including those containing about 25 mg, about 50 mg, about 75 mg, about 100 mg, about 125 mg, about 150 mg, and about 300 mg.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier.

Various other materials may be present as coatings or otherwise modify the physical form of the dosage unit. For instance, tablets, pills, or capsules may be coated with shellac, sugar or both. A syrup or elixir may contain the tetracyclines, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the tetracyclines may be incorporated into sustained-release preparations and formulations. For example, sustained release dosage forms are contemplated wherein the tetracyclines are bound to an ion exchange resin which, optionally, can be coated with a diffusion barrier coating to modify the release properties of the resin or wherein the tetracyclines are associated with a sustained release polymer known in the art, such as hydroxypropylmethylcellulose and the like.

The tetracyclines may also be administered parenterally or intraperitoneally. It is especially advantageous to formulate parenteral compositions in dosage unit form for ease of administration and uniformity of dosage. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersions and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the tetracyclines in the required amount in the appropriate solvent with any of the other ingredients enumerated above, as required, followed by filtered sterilization. Generally, dispersions are prepared by incorporating the various sterilized tetracyclines into a sterile vehicle which contains the basic dispersion medium and the required other ingredients from those enumerated above. In the case of sterile powders, the above solutions are vacuum dried or freeze-dried, as necessary.

The tetracyclines can also be formulated and administered to the patient in solid or liquid particulate form by direct administration, e.g., inhalation, into the respiratory system.

Solid or liquid particulate forms of the tetracyclines prepared for practicing the present invention include particles of respirable size: that is, particles of a size sufficiently small to pass through the mouth and larynx upon inhalation and into the bronchi and alveoli of the lungs. In general, particles ranging from about 1 to 10 microns in size are within the respirable range. The pharmaceutical compositions containing the tetracyclines are preferably administered by direct inhalation into the respiratory system for delivery as a mist or other aerosol or dry powder. Particles of non-respirable size which are included in the aerosol tend to be deposited in the throat and swallowed; thus the quantity of non-respirable particles in the aerosol is preferably minimized.

In the manufacture of the preferred local formulation, in accordance with the description herein, the tetracylines are typically admixed with, inter alia an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation.

Aerosols of liquid particles comprising the tetracyclines may be produced by any suitable means, such as inhalatory delivery systems. One is a traditional nebulizer which works in a mechanism similar to the familiar perfume atomizer. The airborne particles are generated by a jet of air from either a compressor or compressed gas cylinder-passing through the device (pressure driven aerosol nebulizer). In addition, newer forms utilize an ultrasonic nebulizer by vibrating the liquid at a speed of up to about 1 MHz. See, e.g., U.S. Pat. No. 4,501,729, the contents of which are incorporated by reference. Nebulizers are commercially available devices which transform solutions or suspensions of the tetracyclines into a pharmaceutical aerosol mist either by means of acceleration of compressed gas, typically air or oxygen, through a narrow venturi orifice or by means of ultrasonic agitation. Suitable formulations for use in nebulizers consist of the tetracyclines in a liquid carrier. The carrier is typically water (and most preferably sterile, pyrogen-free water) or a dilute aqueous alcoholic solution, preferably made isotonic but may be hypertonic with body fluids by the addition of, for example, sodium chloride. Optional additives include preservatives if the formulation is not made sterile, for example, methyl hydroxybenzoate, as well as antioxidants, flavoring agents, volatile oils, buffering agents and surfactants, which are normally used in the preparation of pharmaceutical compositions.

Aerosols of solid particles comprising the tetracyclines may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the tetracycline or of a powder blend comprising the tetracycline, a suitable powder diluent, such as lactose, and an optional surfactant. A second type of illustrative aerosol generator comprises a metered dose inhaler.

Metered dose inhalers are pressurized aerosol dispensers, typically containing a suspension or solution formulation of the tetracycline in a liquefied propellant. During use, these devices discharge the formulation through a valve, adapted to deliver a metered volume, from about 10 to about 22 microliters to produce a fine particle spray containing tetracycline.

Any propellant may be used in carrying out the present invention, including both chlorofluorocarbon-containing propellants and non-chlorofluorocarbon-containing propellants. Fluorocarbon aerosol propellants that may be employed in carrying out the present invention including fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Examples of such propellants include, but are not limited to: CF₃CHFCF₂, CF₃CH₂CF₂H, CF₃CHFCF₃, CF₃CH₂CF₃, CF₃CHCl—CF₂Cl, CF₃CHCl—CF₃, CF₃CHCl—CH₂Cl, CF₃CHF—CF₂Cl, and the like. A stabilizer such as a fluoropolymer may optionally be included in formulations of fluorocarbon propellants, such as described in U.S. Pat. No. 5,376,359 to Johnson. The aerosol formulation may additionally contain one or more co-solvents, for example, ethanol, surfactants, such as oleic acid or sorbitan trioleate, antioxidants and suitable flavoring agents.

Compositions containing respirable dry particles of micronized tetracyclines may be prepared by grinding the dry active compound, with e.g., a mortar and pestle or other appropriate grinding device, and then passing the micronized composition through a 400 mesh screen to break up or separate out large agglomerates.

The aerosol, whether formed from solid or liquid particles, may be produced by the aerosol generator at a rate of from about 10 to 150 liters per minute. Aerosols containing greater amounts of the tetracycline may be administered more rapidly.

Typically, each aerosol may be delivered to the patient for a period from about 30 seconds to about 20 minutes, with a delivery period of about 1 to 5 minutes being preferred.

The particulate composition comprising the tetracyclines may optionally contain a carrier which serves to facilitate the formation of an aerosol. A suitable carrier is lactose, which may be blended with the tetracycline in any suitable ratio.

The tetracyclines are administered by this mode of administration in therapeutically effective amounts, as defined herein which amounts are determined by the physician.

The tetracyclines can also be applied in therapeutic effective amounts through a transdermal patch using techniques known to one of ordinary skill in the art.

As used herein, “pharmaceutically acceptable carrier” includes any and all solvents, dispersion media, coatings, antibacterial and antifungal agents, isotonic and absorption delaying agents for pharmaceutical active substances well known in the art. Except insofar as any conventional media or agent is incompatible with the tetracyclines, their use in the therapeutic compositions is contemplated. Supplementary active ingredients can also be incorporated into the compositions.

Dosage unit form as used herein refers to physically discrete units suited as unitary dosages for the subjects to be treated; each unit containing a predetermined quantity of active material calculated to produce the desired therapeutic effect in association with the required pharmaceutical carrier.

The tetracycline is compounded for convenient and effective administration in effective amounts with a suitable pharmaceutically acceptable carrier in dosage unit form as hereinbefore described. A unit dosage, for example, contains the principal active cormpound in amounts ranging from about 50 mg to about 1000 mg. If placed in solution, the concentration of the tetracycline preferably ranges from about 10 mg/ml to about 250 mg/ml. The preferred mode of administration is oral. The present inventors, have noted that in normal humans, not suffering from asthma or allergies or diseases wherein IgE is pathogenic, the administration of these tetracyclines does not increase the concentration of IgE in the plasma

The tetracyclines are useful in treating asthma The tetracyclines are anti-inflammatory agents and can be used to treat inflammatory conditions.

The present inventors have found that the higher the concentration of the free IgE in the plasma in those patients suffering from a disease where IgE is pathogenic, the greater is the risk of the disease state becoming aggravated or worsening. By lowering the amount of the free IgE in the plasma of those patients, the physician will lower the risk of the disease state becoming worse or becoming exacerbated.

The effectiveness of the tetracyclines in lowering the concentration of the excess IgE in the plasma can be monitored. For example, the IgE levels in the plasma of the blood, e.g., in peripheral blood, such as peripheral blood mononuclear cells (PBMC), of a patient suffering from a disease wherein IgE is pathogenic, e.g., allergies or asthma especially allergic asthma, e.g., human allergic asthma, inflammatory conditions and the like can be measured prior to treatment. After a period of treatment, the IgE concentration in the plasma is measured again and the change in the concentration of IgE, if any, is noted. If the concentration of the IgE in the plasma is above a threshold level determined by the physician and is not decreased, then the physician should change the treatment regimen and prescribe a greater dose of tetracyclines for the treatment of the disease. If, on the other hand, the concentration of the free IgE is significantly lowered or is less than the threshold level, then the disease is under control, the risk of the disease state worsening is minimized, and the treatment regimen is maintained. In a preferred embodiment, the threshold level is less than about 100 IU/ml and more preferably less than about 75 IU/ml and most preferably less than about 50 IU/ml.

The present inventors have noted that when a patient is suffering from the disease in which IgE is pathogenic, e.g., allergy or asthma, and when the IgE concentration in the serum is greater than 100 IU/ml, there is a substantial risk of the disease worsening. On the other hand, if the IgE concentration in the plasma serum of this patient is less than about 100 IU/ml and more preferably less than about 75 IU/ml after treatment, then there is a lower risk that the disease will worsen.

Thus, using this techniques, the IgE levels of the patient is monitored periodically, e.g., at least about every-three to six months to determine the free IgE level in the plasma and the regimen of treatment reviewed; as indicated hereinabove, if the free concentration of the IgE increases or is above the threshold level, then the physician will increase the dosage and/or change the treatment regimen.

In another embodiment, the present invention is directed to the prophylaxis of the disease wherein IgE is pathogenic from becoming more severe, which method comprises administering to the patient prophylatically effective amount of the tetracyclines, e.g., minocycline or doxycline or combination thereof. The effective amount in this embodiment is determined by the physical usually, this amount the same as the therapeutic effective amount discussed that described hereinabove.

The tetracyclines can also be given to mammals not suffering from asthma, or allergies or other diseases where IgE is pathogenic by administering to the patient prophylactically effective amount of the tetracyclines, e.g., minocycline or doxycycline or combination thereof. The effective amounts can be determined by the physician. As indicated hereinabove, the normal person has a free plasma concentration of IgE of less than about 50 IU/ml. Thus, the administration of the aforementioned tetracyclines will prevent and/or retard the onset of allergy or allergic asthma and/or other disease where IgE is pathogenic. In addition, it will prevent or lower the risk of the persons having a free IgE concentration in the plasma increasing to the level where the person is at a substantial risk of suffering from a disease where IgE is pathogenic. Preferably, the free IgE level in the plasma, after such treatment, will remain less than about 50 IU/ml.

In the present specification, unless indicated to the contrary, the plural will connote the singular and, vice versa.

The term “treating”, “treat” or “treatment”, as used herein refers to the reduction and/or alleviation of at least one adverse effect or symptom of a disease where IgE is pathogenic. It refers to the management and care of a mammalian subject, preferably humans, for the purpose of combating the disease, conditions or disorders where IgE is pathogenic, and includes the administration of the tetracyclines to delay the onset of at least one symptom or complication associated with the disease, alleviating the symptom or effect or complications associated therewith or in the alternative eliminating the disease or condition.

The term “prophylaxis” or “prevent” or synonym thereto refers to the prevention or a measurable reduction in the likelihood of a patient acquiring a disease where IgE is pathogenic, even if the mammal is suffering form another malady which debilitates it and makes it more susceptible to such a disease. If a patient or mammal is suffering from a disease where IgE is pathogenic, the term also refers to the reduction in the likelihood of the disease becoming acerbated.

The term “therapeutically effective amount” is synomous with “IgE lowering effective amounts” and refers to the amount effective or eliminating or alleviating or curing the symptoms associated with a disease or malady where IgE is pathogenic or alleviating or curing the disease altogether.

The term “prophylatically effective amount” refers to the amount effective in preventing or reducing the likelihood of a mammal, e.g., patient from acquiring a disease in which IgE is pathogenic. It also refers to the amount effective in preventing a mammal afflicted with a disease or malady where IgE is pathogenic from worsening or becoming more severe.

As indicated herein, these “amounts” can be determined by the physician; however, it is preferred that these amounts are the same.

The following examples further illustrate the invention.

### EXAMPLES

Patient Specimens

Peripheral blood (40 ml total) was obtained from asthmatic patients (male and female patients, 26-54 yrs. old, mean age 40±14) (n=7) from the SUNY Downstate Asthma Center of Excellence, and non asthmatic controls (n=7). Asthmatic patients presented with clinically defined mild intermittent to severe persistent asthma, with elevated serum IgE levels (>100 IU/ml). Asthma severity was assessed in accordance with National Institutes of Health (NIH) guidelines. Airflow limitation was present in all patients at the time of evaluation. Patients gave informed consent for the use of their blood samples for an experimental study. Control, non asthmatic, subjects (male and female patients, age matched), were recruited from the hospital staff of SUNY DOWNSTATE MEDICAL CENTER who showed no evidence of asthma as identified by history and normal total IgE levels (<50 IU//ml). The study was approved by the institutional review board of the SUNY Downstate Medical Center, Brooklyn, N.Y., and the procedures followed were in accordance with institutional guidelines involving human subjects.

Blood

For studies of serum immunoglobulins, blood was collected into red top Monoject tubes (Sherwood Medical, St. Louis, Mo.) and allowed to clot for 30 min at room temperature. Tubes were rimmed and centrifuged at 1000 rpm for 10 min. Sera were collected and stored at −20° C.

For studies of surface markers, blood was collected into ethylenediamine tetraacetic acid Monoject tubes (Sherwood Medical) and stored at room temperature for up to 2 hr when complete blood counts were performed (Sysmex, McGraw Park, Ill.); flow microfluorimetry studies (Coulter Epics XL/MCL) (Beckman Coulter, Miami, Fla.) were performed within 3 hr.

Determination of Cell Surface Markers

Antibodies

Mouse anti-human monoclonal antibodies (mAbs) directly conjugated to fluoresce in isothiocyanate (FITC) CD45, Simultest CD3/CD4, Simultest CD3/CD8, Simultest CD3/CD19, and appropriately matched isotype control mAbs, were purchased from BD Biosciences (San Jose, Calif.), and used according to manufacturer's recommendation.

Immunoflourescence Assay

Peripheral blood (100 μl) was incubated with conjugated antibodies for 10 min at room temperature. Erythrocytes were lysed with whole blood lysing solution (limunoprep) (Beckman Coulter). Flow cytometric analysis was performed on a Coulter Epics XL/MCL Flow Cytometer using System II software (Coulter) and CytoComp (Coulter). Forward and side scatter were used to identify the lymphocyte population and CD45 was used to establish an optimal lymphocyte gate. A minimum of 15,000 events were collected. The gain on the photomultiplier tube detecting fluorescence intensity was adjusted so that 99% of cells with background fluorescence staining were scored between 10⁰ and 10¹ on a 4-decade log scale. Specific fluorescence was reported as the percentage of cells with relative fluorescence intensity scored above background. Total numbers of cells were calculated from the lymphocyte count. Data-are expressed as total lymphocytes/mm³.

Serum Immunoglobulins

Serum immunoglobulins (IgM, IgG, and IgA) were determined by nephelometry which was performed, according to manufacturer's recommendation (Beckman Array System, Beckman Coulter, Inc.) at the Clinical Immunochemistry Laboratory at SUNY Downstate Medical Center. The results are expressed in mg/dL. (reference range for healthy adult serum: IgM: 60-263 mg/dL; IgG: 694-161 3 mg/dL; IgA: 69-378 mg/dL) Serum IgE levels were detected by the UniCAP Total IgE Fluoroenzyme immunoassay (Pharmacia & Upjohn Diagnostics, Kalamazoo, Mich.) which was performed according to manufacturer's recommendation. To evaluate the test results, the response for the patient's sample was compared directly to the response of the IgE calibrators. Data are expressed as IU/ml (reference range for healthy adult serum: IgE: 20-100 IU/ml).

Cytokine Specific mRNA

RNA extraction and polymerase chain reaction (PCR). Total cellular RNA (2 μg/ml) was extracted from PBMC in accordance with the procedure of Chomczynski, et al. in Anal. Biochem. 1987, 162: 156-159, using Trizol Reagent (GIBCO/BRL), according to manufacturer's recommendation. Pellets were dissolved in TE buffer (10 mM Tris HCl (Sigma, St. Louis, Mo.), pH 7.5, 1 mM EDTA, Sigma), and stored at −70° C. in a Bio-Freezer (Forma Scientific, Marietta, Ohio). Expression of interieukin-2 (IL-2), interieukin-4 (IL-4), interleukin-6 (IL-6), interleukin-10 (IL-10), interferon-gamma (IFN-gamma), interferon-alpha (IFN-alpha), and epsilon specific mRNA was determined using the Advantage One-Step RT-PCR Kit (Clontech, Palo Alto, Calif.), according to manufacturer's recommendation. PCR was conducted using primer pairs specific for IL-2, IL-4, IL-6, IL-10, IFN-gamma, IFN-alpha, and epsilon (Expected band sizes: 305, 344, 628, 328, 427, 303, and 149 basepairs (bp), respectively). A beta-actin primer set was used as an internal positive control. Negative controls, consisting of no mRNA, but addition of primers, were included in every experiment. The PCR amplicons were separated by electrophoresis in a 1.8% agarose (Seakem LE) gel (FMC, Rocldand, Me.), and visualized with ethidium bromide (Sigma).

Cell Cultures

PBMC were separated from blood on a Ficoll-Paque (Pharmacia, Piscataway, N.J.) gradient (density 1.077). The PBMC were carefully removed using a transfer pipette (VWR Scientific, San Francisco, Calif.). Cells were washed twice in RPMI-1640 medium (GIBCO/BRL, Grand Island, N.Y.), resuspended in RPMI (3 ml), and counted on a hemocytometer (Fisher Scientific, Springfield, N.J.). Viability was >98%, as judged by trypan blue exclusion.

Studies of IgE responses induced in vitro were carried out according to the method of Sampson and Buckley in J. Immunol 1981, 127: 829-834. Briefly, PBMC (1.5×10⁶ in 1 ml) were cultured +anti-CD40 mAb (1 ug/ml) (BD Pharmingen, San Diego, Calif.), ± recombinant human interleukin-4 (rhIL-4) (200 U/ml) (Genzyme Corp., Boston, Mass.), in the presence or absence of varying concentrations of either minocycline (Lederle Parenterals Inc., Carolina, Puerto Rico) or doxycycline (American Pharmaceutical Partner Inc., Schaumburg, Ill.), 0.1, 1.0, 10 ug/ml) for 0, 3, and 10 days at 37° C. in complete medium in a humidified 4% CO₂ atmosphere. Cell viability was >98%, as judged by trypan blue exclusion, on day 10.

Antibiotics were reconstituted in sterile dH₂O, according to manufacturer's recommendations. Complete medium contained RPMI-1640 (Fisher Scientific) and was supplemented with N-2-hydroxyethylpiperazine-N′-2-ethanesulfonic acid buffer (HEPES) (25 mM) (Fisher Scientific), penicillin (100 U/ml) (GIBCO/BRL), streptomycin (100 ug/ml) (Eli Lilly and Co., Indianapolis, Ind.), L-glutamine (2 mM) (GIBCO/BRL), and 10% Fetal Calf Serum (FCS) (GIB CO/BRL). The concentration of rhIL-4 in culture was within 1 bog of physiologic relevance (<100 U/ml), according to bioassay data provided by supplier.

For the in vitro quantitative determination of IgE content in cell culture supernatants, solid phase sandwich enzyme linked immunosorbent assays (ELISAs) were performed using IgE ELISA Test Kits (Abexon Trend, Ramsey, Minn.). All ELISAs were performed according to the manufacturer's recommended procedure. Specimens were analyzed in duplicate and a standard curve was derived from known concentrations of IgE. Plates were read using an automated microplate reader (Model ELX800) (Bio-Tek Instruments, Inc., Winooski, Vt.), with a 450-nm measurement filter. Optical densities were converted to IU/ml and/or ng/ml. (1 IU=2.4 ng/IgE protein).

Statistical Analysis

Mixed model ANOVAs were used to compare distributions of lymphocyte subpopulations and serum immunogbobulin levels between asthmatic and non asthmatic groups. The groups were compared on each variable. A P value of <0.05 was considered statistically significant for all comparisons. Statistical analyses were performed using SPSS for Windows version 10.0 software (Chicago, Ill.).

### Results

1. Asthmatic Patients: Medical History and Clinical Features

All asthmatic patients (n=7) exhibited mild intermittent to severe persistent signs or symptoms (>2×/wk) of asthma. Treatment regimens included β-agonist inhalers, inhaled corticosteroids, and if necessary, oral steroids. Table I summarizes the medical history and clinical features of the asthmatic patient.

TABLE I*
ASTHMATIC PATIENTS: MEDICAL HISTORY AND
CLINICAL FEATURES

| | Sex | | Asthma | Signs and | | Positive Skin | Eczema | Serum IgE |
|---|---|---|---|---|---|---|---|---|
| Patient | (Age) | Ethnicity | Classification | Symptoms | Pharmacotherapy | Test | History | (IU/ml)** |
| 1 | F | Hispanic | Severe Persistent | Continual | Inhaled steroids^{a} | (+) | (−) | 355 |
| | (46) | | | | | | | |
| 2 | F | African | Severe Persistent | Continual | Prednisone | (+) | (−) | 719 |
| | (35) | American | | | 10 mg po qd^{b} | | | |
| 3 | M | Caucasian | Mild Intermittent | >2×/wk | Beta Agonist PRN^{c} | (+) | (+) | 241 |
| | (34) | | | | | | | |
| 4 | F | African | Mild Persistent | >2×/wk | Inhaled steroids | (+) | (−) | 209 |
| | (43) | American | | | | | | |
| 5 | M | Caucasian | Mild Intermittent | >2×/wk | Beta Agonist PRN | (+) | (+) | 346 |
| | (45) | | | | | | | |
| 6 | F | Hispanic | Mild Persistent | >2×/wk | Inhaled Steroids | (+) | (−) | 192 |
| | (54) | | | | | | | |
| 7 | F | African | Severe Persistent | Continual | Inhaled Steroids | (+) | (−) | 510 |
| | (26) | American | | | | | | |

*Diagnosis and management of asthma in accordance with National Institutes of Health (NIH) guidelines, July 1997.
**Serum IgE levels of asthmatics (367 ± 51 IU/ml) > non asthmatics (35 ± 51 IU/ml) (P = 0.001); serum 1 gM, IgG and IgA levels in asthmatics (128 ± 19, 1352 ± 140, 309 ± 53 mg/dl, respectively) were similar to non asthmatic controls (127 ± 19, 1223 ± 140, 191 ± 53 mg/dl, respectively). Data are expressed as mg/db or IU/ml + pooled standard error (SE).
^{a}Fluticasone propionate 44 mcg inhalation aerosol: 2 puffs po tid: ter in die (three times daily).
^{b}po: per os (by mouth); qd: quaque die (once daily);
^{c}PRN: pro re nata (when necessary).

Clinical profiles of asthma in this study included: positive skin prick and intradermal test(s) (tree and grass pollens, ragweed pollen, dust mite, American cockroach, Alternaria, Cladosporium, or cat antigens), and/or eczema, and elevated serum IgE (>100 IU/ml) levels. Serum IgE levels were significantly increased in asthmatic (367 IU/ml+51, coefficient of variation [CV]=0.1), compared with control subjects (35 IU/ml+51, CV=1.0; P=0.00 1). In contrast, serum 1 gM, IgG, and IgA (mg/dl) levels in asthmatic patients (128+19, 1352+140, 309+53 mg/dl, respectively) resembled those of controls (127+19, 1223+140, 191+53 mg/dl, respectively). At the time of study, none of the patients were treated with allergen immunotherapy.

2. Distributions of Blood Lymphocyte Subpopulations

Asthmatic and non asthmatic subjects (n=7 per group) had similar total numbers of blood CD3+CD4+ T cells (779/mm³+73, coefficient of variation (CV)=0.09 and 766+115, CV=0.15, respectively) and CD19+ B cells (239+35, CV=0.14 and 379+95/mm³, CV=0.25, respectively). The results are tabulated in Table II.

TABLE II*
DISTRIBUTIONS OF LYMPHOCYTE SUBPOPULATIONS IN PERIPHERAL
BLOOD OF ASTHMATIC AND NON ASTHMATIC HUMANS
Fluorescent cells
Lymphocytes

| | CD3+ CD4+ | | CD3+ CD8+ | | CD19+ | | |
|---|---|---|---|---|---|---|---|
| Subject | mm³ | (%) | mm³ | (%) | mm³ | (%) | CD4/CD8 |
| Asthmatic | 779 ± 73 | 45 ± 3 | 378 ± 66^{a} | 21 ± 3 | 239 ± 35 | 14 ± 2 | 2.06 |
| Non | 766 ± 115 | 36 ± 3 | 568 ± 53^{a} | 28 ± 3 | 379 ± 95 | 17 ± 3 | 1.35 |
| Asthmatic | | | | | | | |

*The distributions of lymphocyte subpopulations in peripheral blood of asthmatic (n = 7) and non asthmatic (n = 7) humans were determined by flow microfluorimetry (Coulter Epics XL/MCL). Data are expressed as mean total cells/mm³ or mean percentage (%) of positive cells ± standard error (SE).
^{a}Asthmatic < non asthmatic (P = 0.045).

The CD3+CD8+ T cell numbers were significantly decreased in the blood of asthmatic patients (378+66/mm³; CV=0.17), compared with non asthmatic subjects (568+53/mm³; CV=0.09; P=0.045). In contrast, the CD4/CD8 ratio was not significantly different between the asthmatic and non asthmatic groups (2.06 and 1.35, respectively).

3. Cytokine Expression by PBMC

On day 0, PBMC from asthmatic patients (17%) expressed IL-2 specific mRNA, compared with non asthmatic subjects (57%). In contrast, PBMC from asthmatic patients (78%) expressed IL-10 specific mRNA, compared with non asthmatic subjects (29%). Neither group expressed IFN-gamma mRNA. However, IL-4, IL-6, and IFN-alpha, and epsilon (data not shown) specific mRNA were similarly expressed by PBMC of both groups, as shown in Table III.

TABLE III*
SUMMARY OF CYTOKINE PRODUCTION BY PERIPHERAL
BLOOD MONONUCLEAR CELLS (PBMC) FROM ASTHMATIC
AND NON ASTHMATIC HUMANS
Cytokines

| | Th-1 | | Th-2 | | | |
|---|---|---|---|---|---|---|
| Subject | IL-2 | IFN-γ | IL-4 | IL-6 | IL-10 | IFN-α |

Asthmatic

| | | | | | | |
|---|---|---|---|---|---|---|
| 1 | − | − | + | − | + | + |
| 2 | − | − | + | + | + | + |
| 3 | + | − | + | + | + | + |
| 4 | − | − | − | + | − | + |
| 5 | nt | − | nt** | nt | + | + |
| 6 | − | − | + | + | + | + |
| 7 | − | − | + | + | − | + |
| Non Asthmatic | | | | | | |
| 1 | − | − | + | − | − | + |
| 2 | − | − | + | + | − | + |
| 3 | + | − | − | − | + | + |
| 4 | + | − | + | + | + | + |
| 5 | − | + | − | − | − | + |
| 6 | + | − | − | − | − | + |
| 7 | + | − | − | + | − | + |

*Unfractionated PBMC from asthmatic and non asthmatic subjects were evaluated for the presence (+) or absence (−) of Th1 type (IL-2, IFN-γ) cytokines, Th2 type (IL-4, IL-6, IL-10) cytokines, and IFN-α. Expression of cytokine-specific mRNA production was determined by Advantage One-Step RT-PCR (Clontech), as described in methods.
nt: not tested.
**nt - not determined

4. Effect of Minocycline or Doxycydline on IgE Responses Induced In vitro

When PBMC from either asthmatic or non asthmatic subjects were cultured with anti-CD40 mAb and rhIL-4, virtually no IgE was detected in culture supernatants on days 0 and 3 (<2.5 ng/ml). In contrast, when asthmatic PBMC (5 of 7 patients) were cultured with anti-CD40 mAb and rh-IL-4, high levels of IgE were detected in supernatants on day 10 (28 ng/ml±12) as shown in Table IV.

TABLE IV*
DOXYCYCLINE OR MINOCYCLINE INHIBITS
IgE PRODUCTION BY STIMULATED PBMC

| | IgE | |
|---|---|---|
| Additions | (ng/ml) | % Inhibition |
| Anti-CD40 + rhIL-4 | 28 ± 12 | 0 |
| Anti-CD40 + rhIL-4 + 0.1 ug doxycycline | 20 ± 9 | 29 |
| Anti-CD40 + rhIL-4 + 1.0 ug doxycycline | 14 ± 7 | 50 |
| Anti-CD40 + rhIL-4 + 10.0 ug doxycycline | 5 ± 2 | 83 |
| Anti-CD40 + rhIL-4 + 0.1 ug minocycline | 16 ± 5 | 43 |
| Anti-CD40 + rhIL-4 + 1.0 ug minocycline | 18 ± 7 | 36 |
| Anti-CD40 + rhIL-4 + 10.0 ug minocycline | 5 ± 2 | 83 |
| *Supernatants were collected from 10 day cultured PBMC (1.5 × 10⁶/ml) stimulated with anti-CD40 mAb (1 ug/ml) plus rhIL-4 (200 U/ml) with or without varying concentrations (0.1, 1.0, 10 ug/ml, respectively) of doxycycline or minocycline. The supernatants were collected and IgE levels were measured by IgE ELISA Test Kit (Alexon Trend). Results are from 5 experiments, repeated 3×, using PBMC obtained from 5 asthmatic subjects. Data are expressed | | |
| # as ng/ml ± standard error (SE). | | |

% Inhibition was calculated as follows:
% Inhibition = 1- (IgE production in each treatment group/IgE production of PBMC stimulated with anti-CD4O mAb plus rhIL-4 without treatment) × 100 (%).

As shown by the data, IgE levels from 2 of 7 asthmatic patients, and all non asthmatic patients did not increase (<2.5 ng/ml). No increases in IgE were detected when PBMC from either asthmatic or non asthmatic subjects were cultured with either anti-CD40 mAb or rhTL-4 alone (<2.5 ng/ml), or with either anti-CD40 mAb or rhIL-4 alone in the presence of either minocycline or doxycline (<2.5 ng/ml).

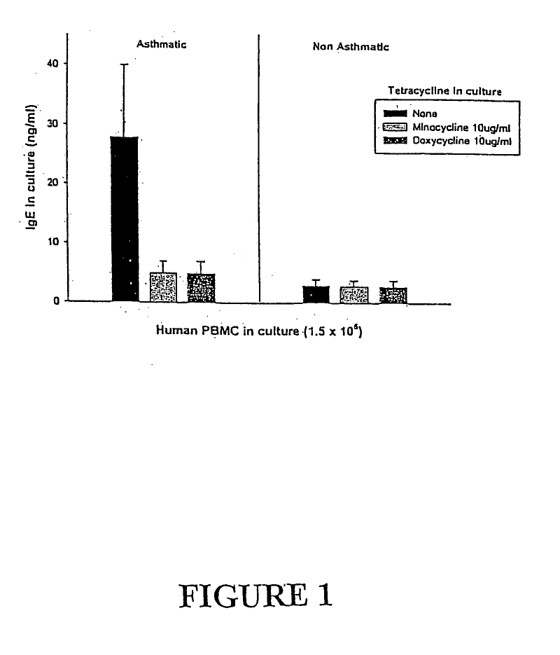

When either minocycline or doxycycline were included in cultures with asthmatic PBMC and anti-CD40 mnAb and rhIL-4, the high levels of IgE obtained on day 10 in the absence of these tetracycline were strongly suppressed, in dose dependent fashion (to >80% with 10 ug/ml), as shown in Table IV. There was no change in IgE levels when either minocycline or doxycycline was included in cultures of non asthmatic PBMC with anti-CD40 mnAb and rhIL-4 (<2.5 ng/ml), (See FIG. 1). More specifically, FIG. 1 illustrates the IgE production in vitro by PMC from asthma and non asthmatic subjects after tetracycline treatment. Human PBMC (1.5×10⁶/1 ml) were obtained from asthmatic and non asthmatic subjects, and cultured ±anti-CD40 mAb (1 ug/ml)+rhIL-4 (200 U/ml), in the presence or absence of minocycline (10 ug/ml) or doxycycline (10 ug/ml) for 10 days. High levels of IgE obtained on day 10 in the absence of tetracyclines were strongly suppressed when tetracyclines were included in cultures of PBMC from asthmatic patients. There was no change in IgE levels when tetracyclines were included in cultures of PBMC from non asthmatic subjects. Results are from 12 experiments, repeated three times, using PBMC obtained from asthmatic subjects (n=5) and non asthmatic subjects (n=7). Data are expressed as ng/ml ± standard error (SE).

As shown, minocycline and doxycycbine suppressed anti-CD40 mAb and rhIL-4 mediated induction of IgE responses by PBMC obtained from asthmatic serum IgE+ atopic donors (asthmatic donors).

From the data, anti-CD40/rhIL-4 induced 5-10 fold higher IgE levels in culture supernatants of PBMC obtained from the asthmatic donors compared with PBMC of non asthmatic, non atopic serum IgE negative donors (non asthmatic donors). This was true despite the fact that there were similar numbers of both CD19+ B cells and CD4+ T cells in cultures of both donor groups (see Table II) on day 0. However, PBMC of the asthmatic donors contained 20-30% fewer CD8+ T cells than those of the non asthmatic donors, with a concomitant increase in other cells in PBMC, all of which have not as yet been identified. Without wishing to be bound it is believed that they include gamma/delta T cells and natural killer cells. It has been reported that gamma/delta T cells downregulate airway responsiveness to allergen challenge. Without wishing to be bound, it is believed that this is effected by controlling the “repair” response of the airway epithelium to alpha/beta T cell mediated damage.

In asthma, both CD4 and CD8 T cell subsets are activated and their numbers are increased in the airways compared to non asthmatic subjects. It has been reported that CD8 T cells are critical in mediating respiratory syncitial virus (RSV)-induced development of lung eosinophilia and airway hyperresponsiveness following allergic airway sensitization in mice. In animal models, it has been reported that CD8+ T cells can attenuate allergic responses including IgE synthesis. Without wishing to be bound CD8+ T cells may provide suppressor function by exerting their various cytokines or through regulation of inflammatory mediators.

The results herein show that asthmatics may exhibit reduced CD8+ T cell suppressor function, thus leading to the perpetuation of CD4+ T cell-mediated inflammation and pro inflammatory (Th2) cytokines. It is believed, without wishing to be bound that the natural immune response to inhaled protein antigens, especially in rats expressing the low IgE responder phenotype, includes a MHC class I-restricted CD8+ T cell component, which is associated with active suppression of IgE antibody production.

The PBMC obtained from asthmatic donors also differed from non asthmatic donors with respect to expression of constitutive IL-2 and-IL-10 mRNA, and in the levels of IgE they secreted in culture. IL-2 mRNA, which was expressed by PBMC of non asthmatic donors (57%) was expressed by fewer asthmatic patients (17%), whereas IL-10 specific mRNA was expressed in the majority of asthmatic patients (78%), compared with non asthmatic donors (29%), implicating the possibility of a reduced Th1-type response and/or an increased Th2-type response in asthmatics. In general, constitutive cytokine specific mRNA expression for IL-4, IL-6, IFN-gamma, IFN-alpha, and epsilon by PBMC was similar for both groups (see Table III). Without wishing to be bound it is believed that Th2-type cytokines play an important role in the immunological processes of allergic asthma. However, the differences in processing and expression of these mRNA transcripts is unknown.

Without wishing to be bound, it is believed that the substantially higher levels of IgE secreted by PBMC of the asthmatic group in response to the nonspecific stimulus, anti-CD40/rhIL-4, show that different regulatory cells such as CD4+ Th2 cells, and macrophages may be present in the asthmatic compared with the non asthmatic PBMC. Although it is well recognized that Th2-type cytokines (IL-4, IL-6, IL-10, respectively) favor IgE responses, and these asthmatic patients had high levels of serum IgE at the time at which their PBMC were obtained (see Table I), there were no detectable differences in mRNA expression of Th2 vs. Th1-type cytokines by PBMC of the asthmatic vs. non asthmatic donors, except for IL-2 and IL-10 mRNA expression. However, other cytokines may have been secreted in vitro to account for the dramatic increase of IgE production by PBMC of the asthmatic group.

The significantly increased IgE production by asthmatics compared with non asthmatic PBMC cannot, however, simply be attributed to increased numbers of either CD19+ B cells, or CD4+ T cells, since similar numbers were present in PBMC of both groups. Without wishing to be bound, it is believed that IL-4 acts as an important inflammatory mediator in asthma IL-4 is also required for induction of IgE responses in vitro.

It is also believed, without wishing to be bound, that another Th2-type cytokine, IL-13, similar to IL-4, can induce IgE synthesis by human B cells. IL-13 induces IgE synthesis by PBMC as well as by purified B cells obtained from PBMC in the presence of activated CD4+ T cell clones. In addition, IL-13 induces B-cell proliferation and differentiation into IgE-secreting cells in the presence of the ligand for CD40, and is believed to contribute to the induction of IgE switching and the maintenance of ongoing IgE synthesis in vivo.

As shown by the exemplification, minocycline and doxycycline suppress anti-CD40/rhIL-4 mediated induction of IgE responses by PBMC of asthmatic donors, in dose dependent fashion, but had no effect on the lower IgE responses elicited by these agents from PBMC of the non asthmatic donors (Figure I).

Thus, minocycline and doxycycline suppressed in vitro induction of IgE responses. Other agents previously shown to suppress IgE responses both in vivo and/or in vitro include: the neuropeptide, substance P, the bacterial cell wall products muramyldipeptide and murabutide, and cytokines such as IL-8, which inhibits IgE production, TGF-Beta, which acts directly on B cells and targets epsilon gennline transcript expression, IFN-alpha, IFN-gamma, and IL-12, which also inhibit IgE secretion.

Thus, in another embodiment of the present invention, these and/or other IgE agents which lower IgE concentrations in the plasma can be used to treat patients suffering from diseases in which IgE is pathogenic and these are administered in effective amounts as defined herein. Moreover, if these are administered to such patients, the effectiveness thereof can be determined by monitoring the free IgE concentration in the plasma in the manner, as described hereinabove.

While the foregoing specification teaches the principle of the present invention, with examples provided for the purpose of illustration, these teachings will make apparent to those skilled in the art other embodiments and examples. These other embodiments and examples are also within the scope of the present invention.

What is claimed is:

1. A method for reducing IgE concentrations in the blood of a patient suffering from a disease comprising administering a therapeutically effective amount of an antibiotic composition.

2. The method of claim 1 wherein said patient is suffering from allergic asthma.

3. The method of claim 1 wherein said antibiotic is a tetracycline.

4. The method of claim 3 wherein said antibiotic is selected from the group consisting of tetracycline, rolitetracycline, oxytetracycline, chlorotetracycline, democlocycline, meclocycline, methacycline, doxycycline and monocycline.

5. The method of claim 3 wherein said antibiotic comprises tetracycline or minocycline.

6. The method of claim 4 wherein said antibiotic comprises a combination of tetracycline and minocycline.

7. The method of claim 4 wherein said antibiotic comprises doxycycline.

8. The method of claim 4 wherein said antibiotic comprises a combination of doxycycline and minocycline.

9. The method of claim 1 wherein said therapeutically effective amount of antibiotic is administered orally.

10. The method of claim 9 wherein said therapeutically effective amount of antibiotic is administered in an amount of about 1 to about 300 mg/day.

11. The method of claim 1 wherein said therapeutically effective amount of antibiotic is administered parenterally.

12. The method of claim 11 wherein said therapeutically effective amount of antibiotic is administered in an amount of about 1 to about 300 mg/day.

13. The method of claim 3 wherein said tetracycline is present in an amount between about 25 mg and about 100 mg.

14. The method of claim 1 wherein said composition further comprises a pharmaceutically acceptable carrier.

15. A method of treating asthma in a patient comprising administering a therapeutically effective amount of an antibiotic composition.

16. The method of claim 15 wherein said antibiotic is a tetracycline.

17. The method of claim 16 wherein said antibiotic is selected from the group consisting of tetracycline, rolitetracycline, oxytetracycline, chlorotetracycline, democlocycline, meclocycline, methacycline, doxycycline and monocycline.

18. The method of claim 16 wherein said antibiotic comprises tetracycline or minocycline.

19. The method of claim 17 wherein said antibiotic comprises a combination of tetracycline and minocycline.

20. The method of claim 17 wherein said antibiotic comprises a combination of doxycycline and minocycline.

21. The method of claim 15, wherein said therapeutically effective amount of antibiotic is administered orally.

22. The method of claim 21 wherein said therapeutically effective amount of antibiotic is administered in an amount of about 1 to about 300 mg/day.

23. The method of claim 15 wherein said therapeutically effective amount of antibiotic is administered parenterally.

24. The method of claim 23 wherein said therapeutically effective amount of antibiotic is administered in an amount of about 1 to about 300 mg/day.

25. The method of claim 16 wherein said tetracycline is present in an amount between about 25 mg and about 100 mg.

26. The method of claim 15 wherein said composition further comprises a pharmaceutically acceptable carrier.

27. A method of monitoring the effectiveness of a drug in lowering the concentration of IgE in the plasma of a mammal suffering from a disease comprising:
a) making an initial determination of the concentration of IgE in the plasma at a first time in said mammal;
b) administering an effective amount of a drug which lowers IgE concentration in the plasma;
c) making a determination of the concentration of IgE in the plasma at a time subsequent to the initial determination; and
d) comparing the values obtained from the first and second determination wherein if the value of the second determination of the free IgE level is higher than or about the same as the first determination and above a threshold level, then the dosage amount of the drug is increased.

28. The method of claim 27 wherein said drug is a tetracycline.

29. The method of claim 28 wherein said tetracycline is minocycline or doxycycline or a combination thereof.
